(19) United States
(12) Patent Application Publication
(10) Pub. No.: US 2019/0076392 A1
(43) Pub. Date: Mar. 14, 2019

(54) METHODS FOR TREATING CANCER

(71) Applicant: Boston Biomedical, Inc., Cambridge, MA (US)

(72) Inventors: Chiang Jia LI, Cambridge, MA (US); Laura BORODYANSKY, Brookline, MA (US)

(21) Appl. No.: 16/070,748

(22) PCT Filed: Jan. 19, 2017
(86) PCT No.: PCT/US2017/014163

§ 371 Date: Jul. 17, 2018

Related U.S. Application Data
(60) Provisional application No. 62/281,004, filed on Jan. 20, 2016.

(51) Int. Cl. A61K 31/337; A61K 31/343; A61K 31/7068; A61P 35/00; A61K 9/00

(52) U.S. Cl. CPC A61K 31/337; A61K 31/343; A61K 9/0019; A61P 35/00; A61K 31/7068

(57) ABSTRACT

Methods of treating cancer using and kits comprising at least one gemcitabine, at least one nab-paclitaxel, and at least one compound of formula (I).

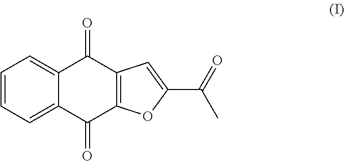 [SMILES: CC(=O)c1cc2c(o1)C(=O)c1ccccc1C2=O]

The present application claims the benefit of priority under 35 U.S.C. § 119 of U.S. Provisional Patent Application No. 62/281,004, filed Jan. 20, 2016; the content of which is hereby incorporated herein by reference in its entirety.

Disclosed herein are methods comprising administering to a subject a combination comprising a therapeutically effective amount of at least one compound of formula (I) in combination with a therapeutically effective amount of at least one gemcitabine chosen from gemcitabine and a therapeutically effective amount of at least one nab-paclitaxel chosen from nab-paclitaxel.

In some embodiments, the at least one compound of formula (I) is chosen from compounds having formula (I)

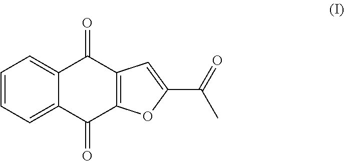 [SMILES: CC(=O)c1cc2c(o1)C(=O)c1ccccc1C2=O]

prodrugs, derivatives, pharmaceutically acceptable salts of any of the foregoing, and solvates of any of the foregoing.

In some embodiments, the at least one compound of formula (I) is chosen from compounds having formula (I)

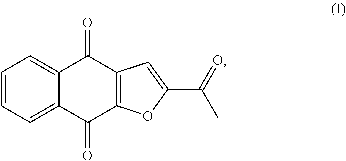 [SMILES: CC(=O)c1cc2c(o1)C(=O)c1ccccc1C2=O]

pharmaceutically acceptable salts thereof, and solvates of any of the foregoing.

Cancer fatalities in the United States alone number in the hundreds of thousands each year. Despite advances in the treatment of certain forms of cancer through surgery, radiotherapy, and chemotherapy, many types of cancer are essentially incurable. Even when an effective treatment is available for a particular cancer, the side effects of such treatment can be severe resulting in a undesirable reduction in a patient's quality of life.

Most conventional chemotherapy agents have significant toxicity and only limited efficacy, particularly for patients with advanced solid tumors. Conventional chemotherapeutic agents cause damage to non-cancerous as well as cancerous cells. The therapeutic index (i.e., a measure of a therapy's ability to discriminate between cancerous and normal cells) of such chemotherapeutic compounds can be quite low. Frequently, a dose of a chemotherapy drug that is effective at killing cancer cells will also kill normal cells, especially those normal cells (such as epithelial cells and cells of the bone marrow) that undergo frequent cell division. When normal cells are affected by the therapy, side effects frequently include hair loss, suppression of hematopoiesis, and nausea. Depending on the general health of a patient, these adverse events can preclude the further administration of chemotherapy, or, at a minimum, subject cancer patients to extremely unpleasant side effects. Even for cancer patients who respond to chemotherapy with tumor regression, cancers often quickly relapse after the initial response to chemotherapy. Such recurrent cancers are often highly resistant or refractory to chemotherapeutics. As discussed below, cancer stem cells (CSCs) or cancer cells with high sternness (sternness-high cancer cells) are responsible for the rapid tumor recurrence and resistance to further traditional chemotherapy.

At least four properties of CSCs are believed to contribute to malignancy: sternness, dysregulation of sternness signaling pathways, a resistance to traditional cancer therapies and a propensity to metastasize.

As used herein, “sternness” generally means the capacity for a stern cell population to self-renew and transform into cancer stern cells (Gupta P B et al., Nat. Med. 2009; 15(9):1010-1012). While CSCs form only a small percentage of the total cancer cell population in a tumor (Clarke M F, Biol. Blood Marrow Transplant. 2009; 11(2 suppl. 2):14-16), they give rise to heterogeneous lineages of differentiated cancer cells that make up the bulk of the tumor (see Gupta et al. 2009). In addition, CSCs possess the ability to spread to other sites in the body by metastasis where they seed the growth of new tumors (Jordan C T et al. N. Engl. J. Med. 2006; 355(12):1253-1261).

The induction and maintenance of sternness properties in CSCs is fueled by a progressive dysregulation of sternness signaling pathways including, but not limited to, those signaling pathways associated with Janus kinase/signal transducers and activators of transcription (JAK/STAT), Hedgehog (Desert (DHH), Indian (IHH), and Sonic (SHH))/PATCHED/(PTCH1)/SMOOTHENED (SMO), NOTCH/DELTA-LIKE (DLL1, DLL3, DLL4)/JAGGED (JAG1, JAG2)/CSL (CBF1/Su(H)/Lag-1), WNT/APC/GSK3/β-CATENIN/TCF4 and NANOG (Boman B M et al., J. Clin. Oncol. 2008; 26(17):2828-2838).

It is the aberrant regulation of these sternness signaling pathways in CSCs (see Boman et al. 2008) that is presumed to confer resistance to chemotherapy and radiation treatment in CSCs which eventually leads to the relapse and spread of the cancer. Thus, while chemotherapy and radiation kills the majority of rapidly dividing bulk cancer cells in a tumor, dysregulation of stemness signaling pathways in CSCs may enable CSCs to avoid chemotherapy induced cell death and also explain how the surviving CSCs acquire the ability to metastasize to sites in the body that are distant from the primary tumor.

The Signal Transducer and Activator of Transcription 3 (also known as Acute-Phase Response Factor, APRF, DNA-Binding Protein APRF, ADMIO 3, HIES; referred to herein as STAT3) is a member of a family of seven ubiquitous transcription factors, STAT1 to STATE, including STAT5a and STAT5b that function at the junction of several cytokine-signaling pathways. For example, STATs can be activated by receptor associated tyrosine kinases like Janus kinases (JAKs) or by receptors with intrinsic tyrosine kinase activity such as, for example, PDGFR, EGFR, FLT3, EGFR, ABL, KDR, c-MET or HER2. Upon tyrosine phosphorylation by receptor associated kinases, the phosphorylated STAT protein (“pSTAT”) dimerizes, as a homo- or heterodimer, and translocates from the cytoplasm to the nucleus, where it binds to specific DNA-response elements in the promoters of target genes and induces gene expression. E.g., FIG. 1. Catlett-Falcone, R., et al. Immunity, 1999. 10(1): p. 105-15; Bromberg, J. F., et al. Cell, 1999. 98(3): p. 295-303; Kanda, N., et al. Oncogene, 2004. 23(28): p. 4921-29; Schlette, E. J., et al. J Clin Oncol, 2004. 22(9): p. 1682-88; Niu, G., et al. Oncogene, 2002. 21(13): p. 2000-08; Xie, T. X., et al. Oncogene, 2004. 23(20): p. 3550-60.

STAT 2, 4, & 6 regulate primarily immune responses, while STAT3, along with STAT1 and STAT5, regulate the expression of genes controlling cell cycle (CYCLIN D1, D2, and c-MYC), cell survival (BCL-XL, BCL-2, MCL-1), and angiogenesis (HIF1α, VEGF) (Furqan et al. Journal of Hematology & Oncology (2013) 6:90). STAT3 is also a key negative regulator of tumor immune surveillance and immune cell recruitment. Kortylewski, M., et al. Nat. Med., 2005. 11(12): p. 1314-21; Burdelya, L., et al. J. Immunol., 2005. 174(7): p. 3925-31; and Wang, T., et al. Nat. Med., 2004. 10(1): p. 48-54.

In normal cells, STAT3 activation is transient and tightly regulated, lasting for example, from about 30 minutes to a few hours. However, in a wide variety of human cancers, including all of the major carcinomas as well as some hematologic tumors, STAT3 is found to be aberrantly active (Lin et al., Oncogene (2000) 19, 2496-2504; Bromberg J. Clin. Invest. (2002) 109:1139-1142; Buettner et al., Clinical Cancer Research (2002) 8, 945-954; Frank Cancer Letters 251 (2007) 199-210 Yu et al. Nature Reviews Cancer (2004) 4, 97-105). Persistently active STAT3 is present in more than half of all breast and lung cancers as well as colorectal cancers (CRC), ovarian cancers, hepatocellular carcinomas, and multiple myelomas and in more than 95% of all head/neck cancers As discussed above, STAT3 is a potent transcription regulator that targets a large number of genes involved in cell cycle, cell survival, oncogenesis, tumor invasion, and metastasis, including, but limited to, BCL-XL, c-MYC, CYCLIN D1, IDO1, PDL1, VEGF, MMP-2, and SURVIVIN. The collective expression of these STAT3 responsive genes maintains the stemness of cancer stem cells (CSCs) required for the survival and propagation of cancer stem cells.

Abrogation of STAT3 signaling using anti-sense oligonucleotides, siRNA, dominant-negative form of STAT3, and/or the targeted inhibition of STAT3 dependent tyrosine kinase activity causes cancer cell-growth arrest, apoptosis, and reduction of metastasis frequency both in vitro and/or in vivo suggesting CSCs stemness is reliant on the constitutive activation of the STAT3 transcription factor. Pedranzini, L., et al. J Clin. Invest., 2004. 114(5): p. 619-22; Bromberg, J. F., et al. Cell, 1999. 98(3): p. 295-303; Darnell, J. E. Nat. Med., 2005. 11(6): p. 595-96; and Zhang, L., et al. Cancer Res, 2007. 67(12): p. 5859-64. STAT3 may therefore play a pivotal role in the survival and self-renewal capacity of CSCs across a broad spectrum of cancers. STAT3 has therefore emerged as a promising target for inhibiting cancer stem cell survival and preventing metastasis. An anti-STAT3 agent with activity against CSCs holds great promise for cancer patients (Boman, B. M., et al. J. Clin. Oncol. 2008. 26(17): p. 2795-99).

As discussed above, CSCs (also called, for example, tumor initiating cells, cancer stem-like cells, stem-like cancer cells, highly tumorigenic cells, or super malignant cells) are a sub-population of cancer cells (found within solid tumors or hematological cancers) that possess characteristics normally associated with stem cells. These cells can grow faster after reduction of non-stem regular cancer cells by chemotherapy, which may be the mechanism responsible for the frequent relapse of cancer after chemotherapies. In contrast to the bulk of cancer cells, which are non-tumorigenic, CSCs are tumorigenic (tumor-forming). In human acute myeloid leukemia, the frequency of these cells is less than 1 in 10,000. Bonnet, D. and J. E. Dick. Nat. Med., 1997. 3(7): p. 730-37. There is mounting evidence to suggest that CSCs exist in almost all tumor types as a distinct population and they give rise to the differentiated cells that form the bulk of the tumor mass and phenotypically characterize the disease. CSCs have been demonstrated to be fundamentally responsible for carcinogenesis, cancer metastasis, cancer recurrence, and relapse. E.g., FIG. 3.

CSCs are inherently resistant to conventional chemotherapies, which means they are left behind by conventional therapies that kill the bulk of tumor cells. e.g., FIG. 2. As such, the existence of CSCs has several implications in terms of cancer treatment and therapy. These include, for example, disease identification, selective drug targets, prevention of cancer metastasis and recurrence, treatment of cancer refractory to chemotherapy and/or radiotherapy, treatment of cancers inherently resistant to chemotherapy or radiotherapy and development of new strategies in fighting cancer.

The efficacy of cancer treatments are, in the initial stages of testing, often measured by the amount of tumor mass they kill off. As CSCs form a very small proportion of the tumor cell population and have markedly different biologic characteristics than their differentiated progeny, the measurement of tumor mass may not select for drugs that act specifically on the stem cells. In fact, CSCs are radio-resistant and refractory to chemotherapeutic and targeted drugs. Normal somatic stem cells are naturally resistant to chemotherapeutic agents—they have various pumps (e.g., multidrug resistance protein pump) that efflux drugs, higher DNA repair capability, and have a slow rate of cell turnover (chemotherapeutic agents naturally target rapidly replicating cells). CSCs, being the mutated counterparts of normal stem cells, may also have similar functions that allow them to survive therapy. In other words, conventional chemotherapies kill differentiated (or differentiating) cells, which form the bulk of the tumor that is unable to generate new cells. E.g., FIG. 2. A population of CSCs that gave rise to the tumor could remain untouched and cause a relapse of the disease.

Furthermore, treatment with chemotherapeutic agents may only leave chemotherapy-resistant CSCs, increasing the likelihood that the ensuing tumor is also resistant to chemotherapy. Cancer stem cells have also been demonstrated to be resistant to radiation therapy (XRT). Hambardzumyan, et al. Cancer Cell, 2006. 10(6): p. 454-56; and Baumann, M., et al. Nat. Rev. Cancer, 2008. 8(7): p. 545-54.

Since surviving CSCs can repopulate the tumor and cause relapse, anti-cancer therapies that include strategies against CSCs hold great promise. Jones R J et al., J Natl Cancer Inst. 2004; 96(8):583-585. By targeting CSC pathways, it may be possible to treat patients with aggressive, non-resectable tumors and refractory or recurrent cancers as well as prevent tumor metastasis and recurrence. Development of specific therapies targeting CSC pathways, therefore, may improve the survival and quality of life of cancer patients, especially those patients suffering from metastatic disease. E.g., FIG. 2. Unlocking this untapped potential may involve the identification and validation of pathways that are selectively important for CSC self-renewal and survival. Though multiple pathways underlying tumorigenesis in cancer and in embryonic stem cells or adult stem cells have been elucidated in the past, pathways for cancer stem cell self-renewal and survival are still sought.

Methods for identification and isolation of CSCs have been reported. The methods used mainly exploit the ability of CSCs to efflux drugs or have been based on the expression of surface markers associated with cancer stem cells.

For example, since CSCs are resistant to many chemotherapeutic agents, it is not surprising that CSCs almost ubiquitously overexpress drug efflux pumps such as ABCG2 (BCRP-1), and other ATP binding cassette (ABC) superfamily members. Ho, M. M., et al. Cancer Res., 2007. 67(10): p. 4827-33; Wang, J., et al. Cancer Res., 2007. 67(8): p. 3716-24; Haraguchi, N., et al. Stem Cells, 2006. 24(3): p. 506-13; Doyle, L. A. and D. D. Ross. Oncogene, 2003. 22(47): p. 7340-58; Alvi, A. J., et al. Breast Cancer Res., 2003. 5(1): p. R1-R8; Frank, N. Y., et al. Cancer Res., 2005. 65(10): p. 4320-33; and Schatton, T., et al. Nature, 2008. 451(7176): p. 345-49. Accordingly, the side population (SP) technique, originally used to enrich hematopoetic and leukemic stem cells, was also employed to identify and isolate CSCs. Kondo, T., et al. Proc. Natl Acad. Sci. USA, 2004. 101(3): p. 781-86. This technique, first described by Goodell et al., takes advantage of differential ABC transporter-dependent efflux of fluorescent dyes such as Hoechst 33342 to define a cell population enriched in CSCs. Doyle, L. A. and D. D. Ross. Oncogene, 2003. 22(47): p. 7340-58; and Goodell, M. A., et al. J. Exp. Med., 1996. 183(4): p. 1797-806. Specifically, the SP is revealed by blocking drug efflux with verapamil, at which point the dyes can no longer be pumped out of the SP.

Efforts have also focused on finding specific markers that distinguish CSCs from the bulk of the tumor. Markers originally associated with normal adult stem cells have been found to also mark CSCs and co-segregate with the enhanced tumorigenicity of CSCs. Commonly expressed surface markers by the CSCs include CD44, CD133, and CD166. Al-Hajj, M., et al. Proc. Natl Acad. Sci. USA, 2003. 100(7): p. 3983-88; Collins, A. T., et al. Cancer Res., 2005. 65(23): p. 10946-51; Li, C., et al. Cancer Res., 2007. 67(3): p. 1030-37; Ma, S., et al. Gastroenterology, 2007. 132(7): p. 2542-56; Ricci-Vitiani, L., et al. Nature, 2007. 445(7123): p. 111-15; Singh, S. K., et al. Cancer Res., 2003. 63(18): p. 5821-28; and Bleau, A. M., et al., Neurosurg. Focus, 2008. 24(3-4): p. E28. Sorting tumor cells based primarily upon the differential expression of these surface marker(s) have accounted for the majority of the highly tumorigenic CSCs described to date. Therefore, these surface markers are validated for identification and isolation of CSCs from the cancer cell lines and from the bulk of tumor tissues.

In some embodiments, the at least one compound of formula (I) is an inhibitor of CSC growth and survival. According to U.S. Pat. No. 8,877,803, the at least one compound of formula (I) inhibits STAT3 pathway activity with a cellular IC50 of ˜0.25 μM. The at least one compound of formula (I) may be synthesized according to U.S. Pat. No. 8,877,803, for example, Example 13. In some embodiments, the at least one compound of formula (I) is used in a method of treating cancers. According to PCT Patent Application No. PCT/US2014/033566, Example 6, the at least one compound of formula (I) was chosen to enter a clinical trial for patients with advanced cancers. The disclosures of U.S. Pat. No. 8,877,803 and PCT Patent Application No. PCT/US2014/033566 are hereby incorporated herein by reference in their entireties for any purpose.

Surprisingly, in clinical trials, patients with higher expression levels of STAT3 showed prolonged overall survival after treatment with at least one compound of formula (I). Thus, the higher the level of pSTAT3 found in a cancer patient before treatment, at least in CRC patients, the higher the overall survival (OS) upon administering a treatment comprising at least one compound of formula (I).

Moreover, a treatment combination of at least one compound of formula (I), at least one gemcitabine, and at least one nab-paclitaxel results in anti-tumor activity with a durable response in patients with metastatic pancreatic ductal adenocarcinoma (mPDAC).

In some embodiments, disclosed herein are methods for treating cancer comprising administering to a subject in need thereof a therapeutically effective amount of at least one compound of formula (I) chosen from compounds having formula (I)

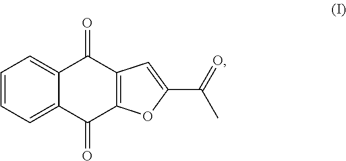 [SMILES: CC(=O)c1cc2c(o1)C(=O)c1ccccc1C2=O]

prodrugs, derivatives, pharmaceutically acceptable salts of any of the foregoing, and solvates of any of the foregoing, a therapeutically effective amount of at least one gemcitabine chosen from gemcitabine, prodrugs, derivatives, pharmaceutically acceptable salt of any of the foregoing, and solvates of any of the foregoing, and a therapeutically effective amount of at least one nab-paclitaxel chosen from nab-paclitaxel, prodrugs, derivatives, pharmaceutically acceptable salt of any of the foregoing, and solvates of any of the foregoing.

In some embodiments, disclosed herein are methods for treating cancer comprising administering to a subject in need thereof a therapeutically effective amount of at least one compound of formula (I), a therapeutically effective amount of at least one gemcitabine, and a therapeutically effective amount of at least one nab-paclitaxel.

The at least one compound of formula (I), the at least one gemcitabine, and the at least one nab-paclitaxel compound may be administered to a patient simultaneously, concurrently, separately, and/or sequentially. Thus, in certain embodiments, the at least one compound of formula (I) and the at least one gemcitabine is administered to a patient simultaneously, concurrently, separately, and/or sequentially. In certain embodiments, the at least one compound of formula (I) and the at least one nab-paclitaxel is administered to a patient simultaneously, concurrently, separately, and/or sequentially.

The at least one compound of formula (I) may be administered daily in a single or a divided dose. The at least one nab-paclitaxel may be administered weekly. The at least one gemcitabine may be administered weekly.

In some embodiments, disclosed herein are methods for sensitizing a subject to at least one therapy regimen comprising administering to a subject in need thereof: a therapeutically effective amount of at least one compound of formula (I) chosen from compounds having formula (I):

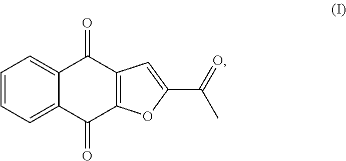 [SMILES: CC(=O)c1cc2c(o1)C(=O)c1ccccc1C2=O]

prodrugs, derivatives, pharmaceutically acceptable salts of any of the foregoing, and solvates of any of the foregoing.

In some embodiments, disclosed herein are methods for sensitizing a subject to at least one therapy regimen comprising administering to a subject in need thereof a therapeutically effective amount of at least one compound of formula (I).

In some embodiments, disclosed herein are methods for resensitizing a subject to at least one prior therapy regimen comprising administering to a subject in need thereof: a therapeutically effective amount of at least one compound of formula (I) chosen from compounds having formula (I):

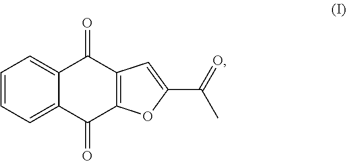 [SMILES: CC(=O)c1cc2c(o1)C(=O)c1ccccc1C2=O]

prodrugs, derivatives, pharmaceutically acceptable salts of any of the foregoing, and solvates of any of the foregoing.

In some embodiments, disclosed herein are methods for resensitizing a subject to at least one prior therapy regimen comprising administering to a subject in need thereof a therapeutically effective amount of at least one compound of formula (I).

In some embodiments, the at least one prior therapy regimen is chosen from chemotherapy regimens. In some embodiments, the at least one prior therapy regimen chosen from gemcitabine regimens. In some embodiments, the at least one prior therapy regimen chosen from taxane chemotherapy regimens.

In some embodiments, disclosed herein are methods for resensitizing a subject to a chemotherapy regimen comprising administering to a subject in need thereof: a therapeutically effective amount of at least one compound of formula (I) chosen from compounds having formula (I):

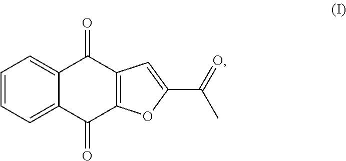 [SMILES: CC(=O)c1cc2c(o1)C(=O)c1ccccc1C2=O]

prodrugs, derivatives, pharmaceutically acceptable salts of any of the foregoing, and solvates of any of the foregoing.

In some embodiments, disclosed herein are methods for resensitizing a subject to a chemotherapy regimen comprising administering to a subject in need thereof a therapeutically effective amount of at least one compound of formula (I).

In some embodiments, a kit is disclosed that comprises at least one compound chosen from compounds having formula (I), prodrugs, derivatives, pharmaceutically acceptable salts of any of the foregoing, and solvates of any of the foregoing. In some embodiments, a kit is disclosed that comprises at least one gemcitabine chosen from gemcitabine, prodrugs, derivatives, pharmaceutically acceptable salts of any of the foregoing, and solvates of any of the foregoing. In some embodiments, a kit is disclosed that comprises at least one nab-paclitaxel chosen from nab-paclitaxel, prodrugs, derivatives, pharmaceutically acceptable salts of any of the foregoing, and solvates of any of the foregoing.

Aspects and embodiments of the present disclosure are set forth or will be readily apparent from the following detailed description. It is to be understood that both the foregoing general description and the following detailed description are exemplary and explanatory only, and are not intended to be restrictive of the claims.

### BRIEF DESCRIPTION OF THE FIGURES

FIG. 1 shows the STAT3 pathway in cancer.

FIG. 2 shows the cancer stem cell specific and conventional cancer therapies.

FIG. 3 shows the formation of heterogeneous cancer cells from cancer stem cells.

FIG. 4 shows an exemplary effect of 2-acetylnaphtho[2,3-b]furan-4,9-dione treatment on p-STAT3 and β-CATENIN protein levels in human colon cancer xenograft tumor (SW480) in nude mice according to certain embodiments of the present disclosure.

FIGS. 5A and 5B show an exemplary effect of a 2-acetylnaphtho[2,3-b]furan-4,9-dione and gemcitabine treatment on pancreatic ductal adenocarcinoma (PDAC) in an xenograft tumor mouse model according to certain embodiments of the present disclosure.

FIG. 6 show an exemplary effect of 2-acetylnaphtho[2,3-b]furan-4,9-dione, gemcitabine (Gemzar), and the combination of 2-acetylnaphtho[2,3-b]furan-4,9-dione and gemcitabine treatment on tumor volume in an xenograft tumor mouse model according to certain embodiments of the present disclosure.

FIG. 7 shows the percent change in target lesions (best response) in patients enrolled in a clinical trial, specifically, in the RP2D determination portion of the clinical trial, according to certain embodiments of the present disclosure. The x-axis shows individual patients.

The following are definitions of terms used in the present specification. The initial definition provided for a group or term herein applies to that group or term throughout the present specification individually or as part of another group, unless otherwise indicated.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In some embodiments, the term “about” is used to modify a numerical value above and below the stated value by a variance of 10%. In some embodiments, the term “about” is used to modify a numerical value above and below the stated value by a variance of 5%. In some embodiments, the term “about” is used to modify a numerical value above and below the stated value by a variance of 1%.

The phrase “and/or,” as used herein in the present teachings and in the claims, should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

When a range of values is listed herein, it is intended to encompass each value and sub-range within that range. For example, “1-5 mg” is intended to encompass 1 mg, 2 mg, 3 mg, 4 mg, 5 mg, 1-2 mg, 1-3 mg, 1-4 mg, 1-5 mg, 2-3 mg, 2-4 mg, 2-5 mg, 3-4 mg, 3-5 mg, and 4-5 mg.

The terms “administer,” “administering,” or “administration” are used herein in their broadest sense. These terms refer to any method of introducing to a subject a compound or pharmaceutical composition described herein and can include, for example, introducing the compound systemically, locally, or in situ to the subject. Thus, a compound of the present disclosure produced in a subject from a composition (whether or not it includes the compound) is encompassed in these terms. When these terms are used in connection with the term “systemic” or “systemically,” they generally refer to in vivo systemic absorption or accumulation of the compound or composition in the blood stream followed by distribution throughout the entire body.

The term “subject” generally refers to an organism to which a compound or pharmaceutical composition described herein can be administered. A subject can be a mammal or mammalian cell, including a human or human cell. The term also refers to an organism, which includes a cell or a donor or recipient of such cell. In various embodiments, the term “subject” refers to any animal (e.g., a mammal), including, but not limited to humans, mammals and non-mammals, such as non-human primates, mice, rabbits, sheep, dogs, cats, horses, cows, chickens, amphibians, reptiles, fish, nematode, and insects, which is to be the recipient of a compound or pharmaceutical composition described herein. Under some circumstances, the terms “subject” and “patient” are used interchangeably herein in reference to a human subject.

The term “therapeutically effective amount” as used herein refers to its meaning as is generally accepted in the art. The term generally refers to the amount of the compound or composition that will elicit the requisite biological or medical response in a cell, tissue, system, animal or human. For example, if a given clinical treatment is considered effective when there is at least about a 25% reduction in a measurable parameter associated with a disease or disorder, a therapeutically effective amount of a drug for the treatment of that disease or disorder is that amount necessary to effect at least about a 25% reduction in that parameter.

A “therapeutically effective amount” in reference to the treatment of cancer, means an amount capable of invoking one or more of the following effects: (1) inhibition, to some extent, of cancer or tumor growth, including slowing down growth or complete growth arrest; (2) reduction in the number of cancer or tumor cells; (3) reduction in tumor size; (4) inhibition (i.e., reduction, slowing down, or complete stopping) of cancer or tumor cell infiltration into peripheral organs; (5) inhibition (i.e., reduction, slowing down, or complete stopping) of metastasis; (6) enhancement of anti-tumor immune response, which may, but is not required to, result in the regression or rejection of the tumor, or (7) relief, to some extent, of one or more measurable symptoms associated with the cancer or tumor. In some embodiments, the “therapeutically effective amount” refers to the amount that is administered systemically, locally, or in situ (e.g., the amount of compound that is produced in situ in a subject). The therapeutically effective amount may vary according to factors such as the disease state, age, sex, and weight of the individual and the ability of one or more anti-cancer agents to elicit a desired response in the individual. A “therapeutically effective amount” is also one in which any toxic or detrimental effects are outweighed by the therapeutically beneficial effects.

Terms such as “treating,” “treatment,” “to treat,” “alleviating,” or “to alleviate” as used herein refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder (“preventing” or “to prevent”). Thus those in need of treatment include those already with the disorder; those prone to have the disorder; and those in whom the disorder is to be prevented.

The term “treating cancer,” “treatment of cancer,” or an equivalent thereof means to decrease, reduce, or inhibit the replication of cancer cells; decrease, reduce, or inhibit the spread (formation of metastases) of cancer; decrease tumor size; decrease the number of tumors (i.e. reduce tumor burden); lessen or reduce the number of cancerous cells in the body; prevent recurrence of cancer after surgical removal or other anti-cancer therapies; and/or ameliorate measurable treatment endpoints (i.e., outcomes).

The term “synergy,” “synergistic,” “synergistically,” or “enhanced” as used herein refers to an effect of interaction or combination of two or more components to produce a combined effect greater than the sum of their separate effects (or “additive effects”).

The term “cancer” refers to the presence of cells possessing characteristics typical of cancer-causing cells, such as uncontrolled proliferation, immortality, metastatic potential, rapid growth and proliferation rate, and certain morphological features. Often, cancer cells will be in the form of a tumor or mass, but such cells may exist alone within a subject, or may circulate in the blood stream as independent cells, such as leukemic or lymphoma cells.

The term “cancer” comprises, for example, AIDS-Related cancers, breast cancers, cancers of the digestive/gastrointestinal tract, endocrine and neuroendocrine cancers, cancers of the eye, genitourinary cancers, germ cell cancers, gynecologic cancers, head and neck cancers, hematologic cancers, musculoskeletal cancers, neurologic cancers, respiratory/thoracic cancers, skin cancers, childhood cancers as well as cancers of unknown primary.

Exemplary AIDS-related cancers include, but are not limited to, AIDS-Related Lymphoma, Primary Central Nervous System Lymphoma and Kaposi Sarcoma.

Exemplary breast cancers include, but are not limited to, ductal carcinomas in situ (DCIS), invasive ductal carcinomas (IDC), invasive lobular carcinoma (ILC), triple negative breast cancers (where the tumor cells are negative for progesterone, estrogen, and HER2/neu receptors), inflammatory breast cancers, metastatic breast cancers, breast cancers during pregnancy, Paget disease of the nipple, Phyllodes tumor, adenoid cystic (or adenocystic) carcinoma, low-grade adenosquamous carcinoma, medullary carcinomas, tubular carcinomas, papillary carcinoma, mucinous (colloid) carcinomas, lymphoma of the breast, adenomyoepithelioma, giant cell sarcoma of the breast, leiomyosarcoma of the breast, angiosarcoma of the breast, cystosarcoma phylloides, and liposarcoma of the breast, carcinoid tumors of the breast, acinic cell carcinoma, oncocytic carcinoma (mammary epithelial oncocytoma), mucoepidermoid carcinoma, spindle cell carcinoma of the breast, squamous cell carcinoma of the breast, secretory carcinoma of the breast (juvenile secretory carcinoma), metaplastic carcinoma of the breast, invasive micropapillary carcinoma of the breast, adenoid cystic carcinoma of the breast, cribriform carcinoma, myofibroblastoma of the breast (benign spindle stromal tumor of the breast) and glycogen-rich clear cell carcinoma of the breast.

Exemplary cancers of the digestive/gastrointestinal tract include, but are not limited to, anal cancer, cancer of the anal region, appendix cancer, gastrointestinal carcinoid tumor, bile duct cancer, carcinoid tumor, gastrointestinal cancer, colon cancer, esophageal cancer, gallbladder cancer, gastrointestinal stromal tumors (GIST), islet cell tumors, pancreatic neuroendocrine tumors, liver cancer, pancreatic cancer, rectal cancer, colorectal adenocarcinoma, small intestine cancer, gastro-esophageal junction (GEJ) cancer, gastric adenocarcinoma and stomach (gastric) cancer.

Exemplary endocrine and neuroendocrine cancers include, but are not limited to, adrenocortical carcinomas, gastrointestinal carcinoid tumors, islet cell tumors, pancreatic neuroendocrine tumors, adrenocortical carcinoma, Merkel cell carcinomas, non-small cell lung neuroendocrine tumors, small cell lung neuroendocrine tumors, parathyroid cancers, pheochromocytomas, pituitary tumors, and thyroid cancers.

Exemplary genitourinary cancers include, but are not limited to, bladder cancer, kidney (renal cell) cancer, penile cancer, prostate cancer, renal pelvis and ureter cancer, transitional cell, testicular cancer, urethral cancer, Wilms tumor and other childhood kidney tumors.

Exemplary gynecologic cancers include, but are not limited to, cervical cancer, endometrial cancer, uterine cancer, fallopian tube cancer, gestational trophoblastic tumor, ovarian epithelial cancer, ovarian germ cell tumor, ovarian low malignant potential tumor, primary peritoneal cancer, uterine sarcoma, vaginal cancer and vulvar cancer.

Exemplary head and neck cancers include, but are not limited to, hypopharyngeal cancer, laryngeal cancer, lip and oral cavity cancer, metastatic squamous neck cancer with occult primary, mouth cancer, nasopharyngeal cancer, oral cavity cancer, lip and oropharyngeal cancer, paranasal sinus and nasal cavity cancer, parathyroid cancer, pharyngeal cancer, salivary gland cancer, throat cancer and thyroid cancer.

Exemplary hematologic cancers include, but are not limited to, leukemias, acute lymphoblastic leukemia, adult, childhood acute lymphoblastic leukemia, adult acute myeloid leukemia, childhood acute myeloid leukemia, chronic lymphocytic leukemia, chronic myelogenous leukemia, hairy cell leukemia, lymphomas, AIDS-related lymphoma, cutaneous T-cell lymphoma, adult Hodgkin lymphoma, childhood Hodgkin lymphoma, Hodgkin lymphoma during pregnancy, mycosis fungoides, childhood Non-Hodgkin lymphoma, adult Non-Hodgkin lymphoma, Non-Hodgkin lymphoma during pregnancy, primary central nervous system lymphoma, Sezary syndrome, cutaneous T-cell lymphoma, Waldenström macroglobulinaemia, chronic myeloproliferative neoplasms, Langerhans cell histiocytosis, multiple myeloma/plasma cell neoplasm, myelodysplastic syndromes and myelodysplastic/myeloproliferative neoplasms.

Exemplary musculoskeletal cancers include, but are not limited to, bone cancer, Ewing's sarcoma, osteosarcoma, malignant fibrous histiocytoma of bone, childhood rhabdomyosarcoma, chondrosarcoma and soft tissue sarcoma.

Exemplary neurologic cancers include, but are not limited to, adult brain tumor, childhood brain tumor, astrocytomas, brain and spinal cord tumors, brain stem glioma, glioblastoma multiforme, atypical teratoid/rhabdoid central nervous system tumor, embryonal central nervous system tumors, germ cell central nervous system tumors, astrocytomas, ependymoma, schwannomas, medulloblastomas, meningiomas craniopharyngioma, neuroblastoma, pituitary tumor, pituitary adenomas and primary central nervous system (CNS) lymphoma.

Exemplary respiratory/thoracic cancers include, but are not limited to, non-small cell lung cancer, small cell lung cancer, malignant mesothelioma, thymoma and thymic carcinoma.

Exemplary skin cancers include, but are not limited to, cutaneous T-cell lymphoma, Kaposi sarcoma, melanoma, Merkel cell carcinoma, skin cancer, cutaneous T-cell lymphoma, mycosis fungoides, intraocular melanoma and Sezary syndrome.

Cancers include refractory versions of any of the above cancers, or a combination of one or more of the above cancers. Some of the exemplified cancers are included in general terms and are included in this term. For example, urological cancer, a general term, includes bladder cancer, prostate cancer, kidney cancer, testicular cancer, and the like; and hepatobiliary cancer, another general term, includes liver cancers (itself a general term that includes hepatocellular carcinoma or cholangiocarcinoma), gallbladder cancer, biliary cancer, or pancreatic cancer. Both urological cancer and hepatobiliary cancer are contemplated by the present disclosure and included in the term “cancer.”

Also included within the term “cancer” is “solid tumor.” As used herein, the term “solid tumor” refers to those conditions, such as cancer, that form an abnormal tumor mass, such as sarcomas, carcinomas, and lymphomas. Examples of solid tumors include, but are not limited to, non-small cell lung cancer (NSCLC), neuroendocrine tumors, thyomas, fibrous tumors, metastatic colorectal cancer (mCRC), and the like. In some embodiments, the solid tumor disease is an adenocarcinoma, squamous cell carcinoma, large cell carcinoma, and the like.

In some embodiments, the cancer is chosen from gastric adenocarcinoma, gastroesophageal junction (GEJ) adenocarcinoma, gastroesophageal adenocarcinoma, non-small cell lung cancer (NSCLC), breast cancer, triple-negative breast cancer (TNBC; i.e., breast cancer that tests negative for estrogen receptors (ER-), progesterone receptors (PR-), and HER2 (Receptor tyrosine-protein kinase erbB-2, also known as CD340 (cluster of differentiation 340), proto-oncogene Neu, ERBB2 (human); HER2-)), ovarian cancer, platinum-resistant ovarian cancer (PROC), pancreatic adenocarcinoma, melanoma, small cell lung cancer, and cholangiocarcinoma. In some embodiments, the cancer is pancreatic adenocarcinoma. In some embodiments, the cancer is pancreatic ductal adenocarcinoma.

Exemplary pancreatic neuroendocrine tumors (pancreatic NETs or PNETs) include, but are not limited to, gastrinomas (Zollinger-Ellison Syndrome), glucagonomas, isulinomas, somatostatinomas, VlPomas (Verner-Morrison Syndrome), Watery Diarrhea and Hypokalemia Achlorhydria (WDHA) Syndrome, nonfunctional islet cell tumors and multiple endocrine neoplasias type-1 (MEN1; also known as Wermer Syndrome).

Exemplary pancreatic exocrine tumors include, but are not limited to, adenocarcinomas, pancreatic ductal adenocarcinomas (PDAC), acinar cell carcinomas, intraductal papillary-mucinous neoplasms (IPMN), mucinous cystadenocarcinomas, solid pseudopapillary neoplasms and pancreatoblastomas.

In some embodiments, each of the cancers is unresectable, advanced, refractory, recurrent, or metastatic.

The terms “progress,” “progressed,” and “progression” as used herein refer to at least one of the following: (1) a response to prior therapy (e.g., chemotherapy) of progressive disease (PD); (2) the appearance of one or more new lesions after treatment with prior therapy (e.g., chemotherapy); and (3) at least a 5% (e.g., 10%, 20%) increase in the sum of diameters of target lesions, taking as a reference the smallest sum on study (this includes the baseline sum if that is the smallest on study).

As used herein, the term “sensitizing” or equivalents thereof (e.g., “sensitize” or “sensitization”) means making subjects that were previously resistant, non-responsive, or somewhat responsive to a therapy regimen (e.g., chemotherapy, targeted therapy, or immunotherapy) sensitive, responsive, or more responsive to that therapy regimen. In certain embodiments, the term “sensitizing” or equivalents thereof includes “resensitizing” or equivalents thereof, making subjects that became resistant, non-responsive, or somewhat responsive to a therapy regimen (e.g., chemotherapy, targeted therapy, or immunotherapy) because of prior exposure to such therapy regimen sensitive, responsive, or more responsive to that therapy regimen.

As used herein, the term “at least one compound of formula (I)” means a compound chosen from compounds having formula (I)

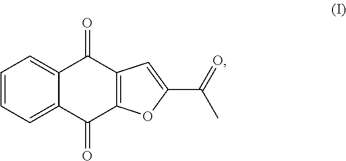 [SMILES: CC(=O)c1cc2c(o1)C(=O)c1ccccc1C2=O]

prodrugs, derivatives, pharmaceutically acceptable salts of any of the foregoing, and solvates of any of the foregoing.

In some embodiments, prodrugs or derivatives of compounds having formula (I) are STAT3 inhibitors. Non-limiting examples of prodrugs of compounds having formula (I) are the phosphoric ester and phosphoric diester described in U.S. pre-grant Publication No. 2012/0252763 as compound numbers 4011 and 4012 and also suitable compounds described in in U.S. Pat. No. 9,150,530. Non-limiting examples of derivatives of compounds having formula (I) include the derivatives disclosed in U.S. Pat. No. 8,977,803. The disclosures of U.S. pre-grant Publication No. 2012/0252763 and U.S. Pat. Nos. 9,150,530 and 8,977,803 are hereby incorporated herein by reference in their entireties for any purpose. In certain embodiments, the term “at least one compound of formula (I)” means a compound chosen from compounds having formula (I)

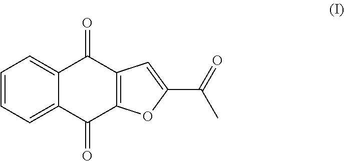 [SMILES: CC(=O)c1cc2c(o1)C(=O)c1ccccc1C2=O]

pharmaceutically acceptable salts of any of the foregoing, and solvates of any of the foregoing.

Compounds having formula (I), shown below,

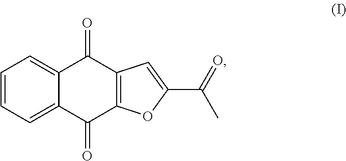 [SMILES: CC(=O)c1cc2c(o1)C(=O)c1ccccc1C2=O]

may also be known as 2-acetylnaphtho[2,3-b]furan-4,9-dione, napabucasin, or 661608 and include tautomers thereof.

Suitable methods of preparing 2-acetylnaphtho[2,3-b]furan-4,9-dione, including its crystalline forms and additional cancer stemness inhibitors, are described in the co-owned PCT applications published as WO 2009/036099, WO 2009/036101, WO 2011/116398, WO 2011/116399, and WO 2014/169078; the content of each application is hereby incorporated herein by reference in its entirety for any purpose.

As used herein, the term “at least one gemcitabine” means a compound chosen from gemcitabine, prodrugs, derivatives, pharmaceutically acceptable salt of any of the foregoing, and solvates of any of the foregoing. In certain embodiments, the term “at least one gemcitabine” means a compound chosen from gemcitabine, pharmaceutically acceptable salt of any of the foregoing, and solvates of any of the foregoing.

As used herein, the term “at least one nab-paclitaxel” means a compound chosen from nab-paclitaxel, prodrugs, derivatives, pharmaceutically acceptable salt of any of the foregoing, and solvates of any of the foregoing. In certain embodiments, the term “at least one nab-paclitaxel” means a compound chosen from nab-paclitaxel, pharmaceutically acceptable salt of any of the foregoing, and solvates of any of the foregoing.

The term “salt(s),” as used herein, includes acidic and/or basic salts formed with inorganic and/or organic acids and bases. As used herein, the term “pharmaceutically acceptable salt” refers to those salts which are, within the scope of sound medical judgment, suitable for use in contact with the tissues of subjects without undue toxicity, irritation, allergic response and/or the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19.

Pharmaceutically acceptable salts may be formed with inorganic or organic acids. Non-limiting examples of suitable inorganic acids include hydrochloric acid, hydrobromic acid, phosphoric acid, sulfuric acid, and perchloric acid. Non-limiting examples of suitable organic acids include acetic acid, oxalic acid, maleic acid, tartaric acid, citric acid, succinic acid, and malonic acid. Other non-limiting examples of suitable pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid.

Salts may be prepared in situ during the isolation and purification of the disclosed compound, or separately, such as by reacting the compound with a suitable base or acid, respectively. Non-limiting examples of pharmaceutically acceptable salts derived from bases include alkali metal, alkaline earth metal, ammonium and N⁺(C₁₋₄alkyl)₄ salts. Non-limiting examples of suitable alkali or alkaline earth metal salts include sodium, lithium, potassium, calcium, magnesium, iron, zinc, copper, manganese, and aluminum salts. Further non-limiting examples of suitable pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Non-limiting examples of suitable organic bases from which salts may be derived include primary amines, secondary amines, tertiary amines, substituted amines including naturally occurring substituted amines, cyclic amines, and basic ion exchange resins, such as isopropylamine, trimethylamine, diethylamine, triethylamine, tripropylamine, and ethanolamine. In some embodiments, pharmaceutically acceptable base addition salts can be chosen from ammonium, potassium, sodium, calcium, and magnesium salts.

The term “solvate” represents an aggregate that comprises one or more molecules of a compound of the present disclosure with one or more molecules of a solvent or solvents. Solvates of the compounds of the present disclosure include, for example, hydrates.

In some embodiments, gemcitabine and nab-paclitaxel are administered according to a regimen on days 1, 8, and 15 of every 28 day cycle. In some embodiments, gemcitabine (e.g., about 1000 mg/m² or a fraction (e.g., 25%, 50%, 75%, or 90%) thereof) is administered weekly. In some embodiments, gemcitabine (e.g., about 1000 mg/m² or a fraction (e.g., 25%, 50%, 75%, or 90%) thereof) is administered weekly up to 7 weeks. In some embodiments, gemcitabine (e.g., about 1000 mg/m² or a fraction (e.g., 25%, 50%, 75%, or 90%) thereof) is administered weekly for 3 out of every 4 weeks. In some embodiments, nab-paclitaxel (e.g., about 100 mg/m², about 125 mg/m², about 250 mg/m², or about 260 mg/m²) is administered weekly. In some embodiments, nab-paclitaxel (e.g., about 100 mg/m², about 125 mg/m², about 250 mg/m², or about 260 mg/m²) is administered weekly up to 7 weeks. In some embodiments, nab-paclitaxel (e.g., about 100 mg/m², about 125 mg/m², about 250 mg/m², or about 260 mg/m²) is administered weekly for 3 out of every 4 weeks. In some embodiments, nab-paclitaxel (e.g., about 100 mg/m², about 125 mg/m², about 250 mg/m², or about 260 mg/m²) is administered every 3 weeks.

The at least one compound disclosed herein may be in the form of a pharmaceutical composition. In some embodiments, the pharmaceutical compositions may comprise the at least one compound of formula (I) and at least one pharmaceutically acceptable carrier. In some embodiments, the pharmaceutical compositions may comprise one or more compounds and at least one pharmaceutically acceptable carrier, where the one or more compounds are capable of being converted into the at least one compound of formula (I) in a subject (i.e., a prodrug).

The term “carrier” as used herein means a pharmaceutically acceptable material, composition or vehicle, such as, for example, a liquid or solid filler, diluent, excipient, solvent or encapsulating material involved in or capable of carrying or transporting the subject pharmaceutical compound from one organ, or portion of the body, to another organ, or portion of the body. Each carrier must be “acceptable” in the sense of being compatible with the other ingredients of the formulation and not injurious to the patient. Non-limiting examples of pharmaceutically acceptable carriers, carriers, and/or diluents include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers, and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

In some embodiments, the at least one compound of formula (I) may be administered in an amount ranging from about 80 mg to about 1500 mg. In some embodiments, the at least one compound of formula (I) may be administered in an amount ranging from about 160 mg to about 1000 mg. In some embodiments, the at least one compound of formula (I) may be administered in an amount ranging from about 300 mg to about 700 mg. In some embodiments, the at least one compound of formula (I) may be administered in an amount ranging from about 700 mg to about 1200 mg. In some embodiments, the at least one compound of formula (I) may be administered in an amount ranging from about 800 mg to about 1100 mg. In some embodiments, the at least one compound of formula (I) may be administered in an amount ranging from about 850 mg to about 1050 mg. In some embodiments, the at least one compound of formula (I) may be administered in an amount ranging from about 960 mg to about 1000 mg. In some embodiments, the total amount of the at least one compound of formula (I) is administered once daily. In some embodiments, the at least one compound of formula (I) is administered in a dose of about 480 mg daily. In some embodiments, the at least one compound of formula (I) is administered in a dose of about 960 mg daily. In some embodiments, the at least one compound of formula (I) is administered in a dose of about 1000 mg daily. In some embodiments, the total amount of the at least one compound of formula (I) is administered in divided doses (more than once) daily, such as twice daily (BID) or more often. In some embodiments, the at least one compound of formula (I) may be administered in an amount ranging from about 80 mg to about 750 mg twice daily. In some embodiments, the at least one compound of formula (I) may be administered in an amount ranging from about 80 mg to about 500 mg twice daily. In some embodiments, the at least one compound of formula (I) is administered in a dose of about 240 mg twice daily. In some embodiments, the at least one compound of formula (I) is administered in a dose of about 480 mg twice daily. In some embodiments, the at least one compound of formula (I) is administered in a dose of about 500 mg twice daily.

Pharmaceutical compositions disclosed herein that are suitable for oral administration may be in the form of capsules, cachets, pills, tablets, lozenges (using a flavored basis, usually sucrose and acacia or tragacanth), powders, granules, a solution in an aqueous or non-aqueous liquid, a suspension in an aqueous or non-aqueous liquid, an oil-in-water emulsion, a water-in-oil emulsion, an elixir, a syrup, pastilles (using an inert base, such as gelatin, glycerin, sucrose, and/or acacia) and/or mouthwashes, each containing a predetermined amount of the at least one compound of the present disclosure.

A pharmaceutical composition disclosed herein may be administered as a bolus, electuary, or paste.

Solid dosage forms for oral administration (capsules, tablets, pills, dragees, powders, granules and the like) may be mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polypropylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type also may be employed as fillers in soft and hard-filled gelatin capsules using such excipients as lactose or milk sugars, as well as high molecular weight polyethylene glycols and the like.

Liquid dosage forms for oral administration may include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxypropyl-3-cyclodextrin, may be used to solubilize compounds.

The pharmaceutical compositions also may include adjuvants such as wetting agents, emulsifying and suspending agents, sweetening, flavoring, coloring, perfuming and preservative agents. Suspensions, in addition to the compounds according to the disclosure, may contain suspending agents as, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions disclosed herein, for rectal or vaginal administration may be presented as a suppository, which may be prepared by mixing one or more compounds according to the present disclosure, with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the compounds of the present disclosure. Pharmaceutical compositions which are suitable for vaginal administration also may include pessaries, tampons, creams, gels, pastes, foams or spray formulations containing carriers that are known in the art to be appropriate.

Dosage forms for the topical or transdermal administration of a pharmaceutical composition or pharmaceutical tablet of the present disclosure may include powders, sprays, ointments, pastes, creams, lotions, gels, solutions, patches and inhalants. The pharmaceutical composition or pharmaceutical tablet may be mixed under sterile conditions with a pharmaceutically acceptable carrier, and with any preservatives, buffers, or propellants which may be required.

The ointments, pastes, creams and gels may contain, in addition to the pharmaceutical composition or pharmaceutical tablet of the present disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a pharmaceutical composition or a pharmaceutical tablet of the present disclosure, excipients such as lactose, talc, silicic acid, aluminum hydroxide, calcium silicates and polyamide powder, or mixtures of these substances. Additionally, sprays may contain customary propellants, such as chlorofluorohydrocarbons and volatile unsubstituted hydrocarbons, such as butane and propane.

Ophthalmic formulations, eye ointments, powders, solutions and the like, are also contemplated as being within the scope of the present disclosure.

Compositions suitable for parenteral administration may comprise at least one more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In various embodiments, a composition described herein includes at least one compound of formula (I) and one or more surfactants. In some embodiments, the surfactant is sodium lauryl sulfate (SLS), sodium dodecyl sulfate (SDS), or one or more polyoxylglycerides. For example, the polyoxyglyceride can be lauroyl polyoxylglycerides (sometimes referred to as Gelucire™) or linoleoyl polyoxylglycerides (sometimes referred to as Labrafil™). Examples of such compositions are shown in PCT Patent Application No. PCT/US2014/033566, the contents of which are incorporated herein in its entirety.

As noted above, the methods disclosed herein may treat at least one disorder related to aberrant STAT3 pathway activity in a subject. Aberrant STAT3 pathway activity can be identified by expression of phosphorylated STAT3 (“pSTAT3”), or its surrogate upstream or downstream regulators or through the detection of pSTAT3 localized to the nucleus.

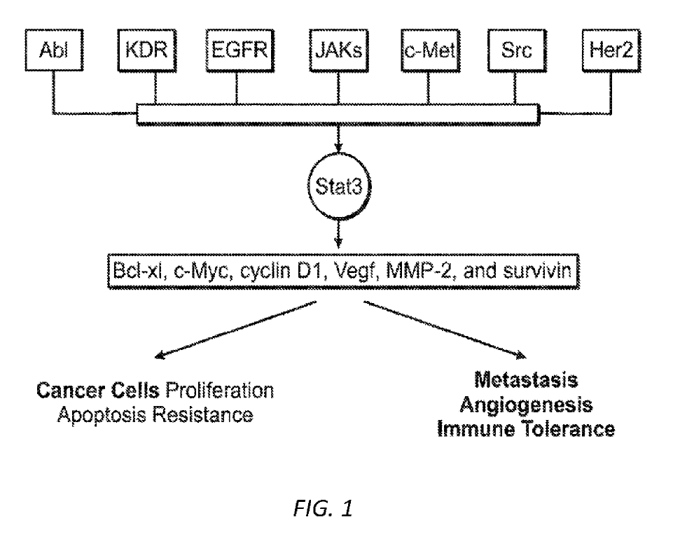

The STAT3 pathway can be activated in response to cytokines, for example, IL-6, or by one or more tyrosine kinases, for example, EGFR, JAKs, ABL, KDR, c-MET, SRC, and HER2. See, e.g., FIG. 1. The downstream effectors of STAT3 include, but are not limited to, BCL-XL, c-MYC, CYCLIND1, VEGF, MMP-2, and SURVIVIN. Id. The STAT3 pathway has been found to be aberrantly active in a wide variety of cancers, as shown in Table 1. Persistently active STAT3 pathway may occur in more than half of breast and lung cancers, hepatocellular carcinomas, multiple myelomas and in more than 95% of head and neck cancers. Blocking the STAT3 pathway causes cancer cell-growth arrest, apoptosis, and reduction of metastasis frequency in vitro and/or in vivo. Activated STAT3 has also been demonstrated in a number of autoimmune and inflammatory diseases. Furthermore, as interleukin-6 mediated inflammation has been disclosed to be the common causative origin for Atherosclerosis, Peripheral Vascular Disease, Coronary Artery Disease, hypertension, Osteroprorosis, Type 2 Diabetes, and Dementia, and as gp130-JAKS-STATs has been disclosed to be the main pathway activated by IL-6, inhibition of the STAT3 pathway may treat or prevent these diseases as well. Libby, P., et al. Circulation, 2002. 105(9): p. 1135-43; Stephens, J. W., et al. Mol. Genet. Metab., 2004. 82(2): p. 180-86; Cesari, M., et al. Circulation, 2003. 108(19): p. 2317-22; Orshal, J. M. and R. A. Khalil. Am. J. Physiol. Regul. Integr. Comp. Physiol., 2004. 286(6): p. R1013-23; Manolagas, S. C. Bone, 1995. 17(2 Suppl): p. 63S-67S; and Yaffe, K., et al. Neurology, 2003. 61(1): p. 76-80.

TABLE 1
DISEASES

| | | |
|---|---|---|
| ONCOLOGY | Solid tumors | Breast Cancer (Watson, C. J. and W. R. Miller. |
| DISEASES | | Br. J. Cancer, 1995. 71(4): p. 840-44) |
| | | Head and Neck Cancer (SCCHN) (Song, J. I. |
| | | and J. R. Grandis. Oncogene, 2000. 19(21): p. |
| | | 2489-95) |
| | | Lung Cancer (Song, L., et al. Oncogene, 2003. |
| | | 22(27): p. 4150-65) |
| | | Ovarian Cancer (Savarese, T. M., et al. |
| | | Cytokine, 2002. 17(6): p. 324-34) |
| | | Pancreatic Cancer (Toyonaga, T., et al. |
| | | Cancer Lett., 2003. 201(1): p. 107-16) |
| | | Colorectal carcinoma (Corvinus, F. M., et al. |
| | | Neoplasia, 2005. 7(6): p. 545-55) |
| | | Prostate Cancer (Gao, B., et al. FEBS Lett., |
| | | 2001. 488(3): p. 179-84) |
| | | Renal Cell carcinoma (Buettner, R., et al. Clin. |
| | | Cancer Res., 2002. 8(4): p. 945-54) |
| | | Melanoma (Carson, W. E. Clin. Cancer Res., |
| | | 1998. 4(9): p. 2219-28) |
| | | Hepatocellular carcinomas (Darnell, J. E. Nat. |
| | | Med., 2005. 11(6): p. 595-96) |
| | | Cervical Cancer (Chen, C. L., et al. Br. J. |
| | | Cancer, 2007. 96(4): p. 591-99) |
| | | Endometrial Cancer (Chen, C. L., et al. Br. J. |
| | | Cancer, 2007. 96(4): p. 591-99) |
| | | Sarcomas (Lai, R., et al. J. Pathol., 2006. |
| | | 208(5): p. 624-32; and) |
| | | Brain Tumors (Punjabi, A. S., et al. J. Virol., |
| | | 2007. 81(5): p. 2449-58) |
| | | Gastric Cancers (Kanda, N., et al. Oncogene, |
| | | 2004. 23(28): p. 4921-29) |
| | Hematologic | Multiple Myeloma (Puthier, D., et al. Eur. J. |
| | Tumors | Immunol., 1999. 29(12): p. 3945-50) |

| | |
|---|---|
| Leukemia | HTLV-1-dependent Leukemia |
| | (Migone, T. S., et al. Science, |
| | 1995. 269(5220): p. 79-81) |
| | Chronic Myelogenous Leukemia |
| | (Buettner, R., et al. Clin. Cancer |
| | Res., 2002. 8(4): p. 945-54) |
| | Acute Myelogenous Leukemia |
| | (Spiekermann, K., et al. Eur. J. |
| | Haematol., 2001. 67(2): p. 63-71) |
| | Large Granular Lymphocyte |
| | Leukemia (Epling-Burnette, P. |
| | K., et al. J. Clin. Invest., 2001. |
| | 107(3): p. 351-62) |
| Lymphomas | EBV-related/Burkitt's (Weber- |
| | Nordt, R. M., et al. Blood, 1996. |
| | 88(3): p. 809-16) |
| | Mycosis Fungoides (Buettner, |
| | R., et al. Clin. Cancer Res., |
| | 2002. 8(4): p. 945-54) |
| | HSV Saimiri-dependent (T-cell) |
| | (Buettner, R., et al. Clin. Cancer |
| | Res., 2002. 8(4): p. 945-54) |
| | Cutaneous T-cell Lymphoma |
| | (Sommer, V. H., et al. Leukemia, |
| | 2004. 18(7): p. 1288-95) |
| | Hodgkin's Diseases (Buettner, |
| | R., et al. Clin. Cancer Res., |
| | 2002. 8(4): p. 945-54) |
| | Anaplastic Large-cell Lymphoma |
| | (Lai, R., et al. Am. J. Pathol., |
| | 2004. 164(6): p. 2251-58) |

| | | |
|---|---|---|
| IMMUNE | Inflammatory | Inflammatory Bowel Diseases (Fu, X. Y. Cell |
| DISEASES | Diseases | Res., 2006. 16(2): p. 214-19) |
| | | Inflammatory Arthritis (Feldmann, M., et al. |
| | | Ann. Rev. Immunol., 1996. 14: p. 397-440; |
| | | Krause, A., et al. J. Immunol, 2002. 169(11): p. |
| | | 6610-16; Pfitzner, E., et al. Curr. Pharm. Des., |
| | | 2004. 10(23): p. 2839-50) |
| | | Crohn's Diseases (Lovato, P., et al. J. Biol. |
| | | Chem., 2003. 278(19): p. 16777-81) |
| | | Chronic inflammatory conditions (Ishihara, K. |
| | | and T. Hirano. Cytokine Growth Factor Rev., |
| | | 2002. 13(4-5): p. 357-68) |
| | Autoimmune | Rheumatoid Arthritis (Feldmann, M., et al. Ann. |
| | | Rev. Immunol., 1996. 14: p. 397-440; Krause, |
| | | A., et al. J. Immunol, 2002. 169(11): p. 6610- |
| | | 16; Ivashkiv, L. B. and I. Tassiulas. J. Clin. |
| | | Invest., 2003. 111(6): p. 795-97; Sengupta, T. |
| | | K., et al. J. Exp. Med., 1995. 181(3): p. 1015- |
| | | 25; and Shouda, T., et al. J. Clin. Invest., 2001. |
| | | 108(12): p. 1781-88) |
| | | Systemic lupus (Harada, T., et al. |
| | | Autoimmunity, 2007. 40(1): p. 1-8) |

| | |
|---|---|
| | Asthma (Simeone-Penney, M. C., et al. J. Immunol., 2007. |
| | 178(10): p. 6191-99) |
| | Allergy (Hagler, M., et al. J. of Allergy Clin. Immunol., 2007. |
| | 119(S1): p. S263-S265) |
| | Infections (Benkhart, E. M., et al. J. Immunol., 2000. 165(3): p. |
| | 1612-17) |
| PROLIFERATIVE | Psoriasis (Sano, S., et al. Nat. Med., 2005. 11(1): p. 43-49) |
| DISORDERS | Keloids (Lim, C. P., et al. Oncogene, 2006. 25(39): p. 5416-25) |
| | Warts (Arany, I., et al. Antimicrob. Agents Chemother., 2000. |
| | 44(7): p. 1869-73) |
| | Myelodysplastic syndrome (Tefferi, A. Hematology Am. Soc. |
| | Hematol. Educ. Program, 2006: p. 240-45) |
| | Polycynthemia vera (Roder, S., et al. Exp. Hematol., 2001. 29(6): |
| | p. 694-702) |
| CNS | Alzheimer's Disease (Kim, O, S., et al. J. Biol. Chem., 2002. |
| DISEASES | 277(43): p. 40594-601; Wyss-Coray, T. Nat. Med., 2006. 12(9): p. |
| | 1005-15; and Campbell, I. L. Brain Res. Rev., 2005. 48(2): p. 166- |
| | 77) |
| | Multiple Sclerosis (MS) (Kim, O, S., et al. J. Biol. Chem., 2002. |
| | 277(43): p. 40594-601; Campbell, I. L. Brain Res. Rev., 2005. |
| | 48(2): p. 166-77; and Stelmasiak, Z., et al. Med. Sci. Monit., 2000. |
| | 6(6): p. 1104-08) |

In some embodiments, the at least one disorder may be chosen from cancers having aberrant STAT3 pathway activity. For example, activated pSTAT3 has been detected in pancreatic cancer cells (Wei et al. Oncogene (2003) 22(3): 319-329; Scholz et al. Gastroenterology (2003) 125:891-905; Toyonaga et al. Cancer Lett. (2003) 10; 201(1):107-16; Qiu et al. Cancer Sci. (2007) 98(7):1099-106).

In some embodiments, the at least one disorder may be chosen from autoimmune diseases related to aberrant STAT3 pathway activity and inflammatory diseases related to aberrant STAT3 pathway activity. In some embodiments, the diseases related to aberrant STAT3 pathway activity may be chosen from inflammatory bowel diseases, arthritis, Crohn's diseases, ulcerative colitis, rheumatoid arthritis, asthma, allergy, and systemic lupus erythematosus.

In some embodiments, the at least one disorder may be chosen from CNS diseases related to aberrant STAT3 pathway activity. In some embodiments, the CNS diseases may be chosen from autoimmune demyelination disorders, Alzheimer's, strokes, ischemia reperfusion injuries, and multiple sclerosis. In some embodiments, the at least one disorder is chosen from diseases caused by inflammation and related to aberrant STAT3 pathway activity. In some embodiments, the diseases caused by inflammation and related aberrant STAT3 pathway activity may be chosen from peripheral vascular disease, coronary artery disease, hypertension, osteoporosis, type 2 diabetes, and dementia.

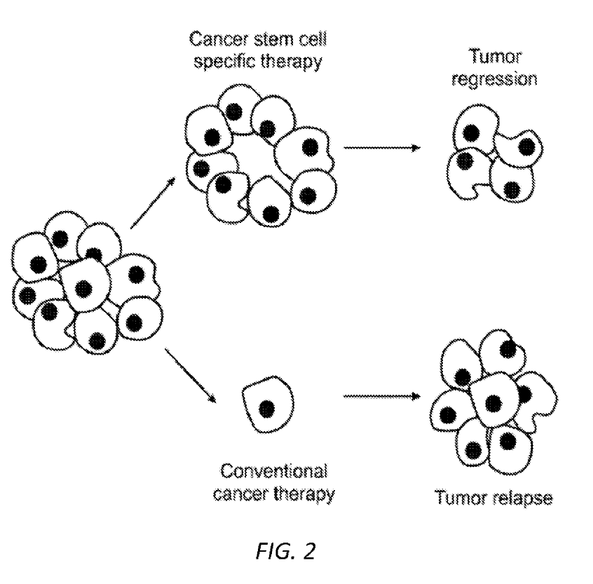

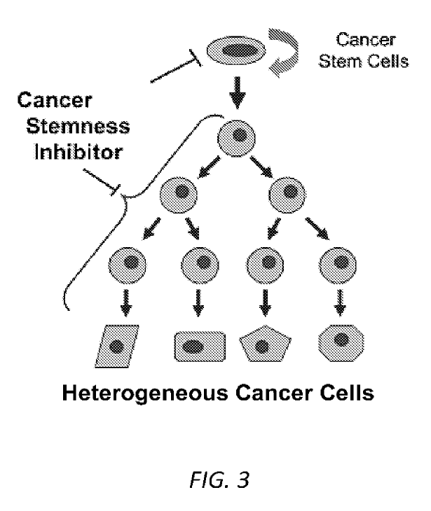

Recent studies have disclosed cancer stem cells able to regenerate tumors. See, e.g., FIG. 3. These cancer stem cells are disclosed to be functionally linked with continued malignant growth, cancer metastasis, recurrence, and cancer drug resistance. Cancer stem cells and their differentiated progeny appear to have markedly different biologic characteristics. They persist in tumors as a distinct, but rare population. Conventional cancer drug screenings depend on measurement of the amount of tumor mass and, therefore, may not identify drugs that act specifically on the stem cells. In fact, cancer stem cells have been disclosed to be resistant to standard chemotherapies and are enriched after standard chemotherapy treatments, see, e.g., FIG. 2, which can result in refractory cancer and recurrence. Cancer stem cells have also been demonstrated to be resistant to radiotherapy. Baumann, M., et al. Nat. Rev. Cancer, 2008. 8(7): p. 545-54. The reported cancer types in which cancer stem cells have been isolated include breast cancer, head cancer, neck cancer, lung cancer, ovarian cancer, pancreatic cancer, colorectal carcinoma, prostate cancer, melanoma, multiple myeloma, Kaposi sarcoma, Ewing's sarcoma, liver cancer, medulloblastoma, brain tumors, and leukemia. STAT3 has been identified as a cancer stem cell survival and self-renewal factor. Therefore, STAT3 inhibitors may kill cancer stem cells and/or may inhibit cancer stem cell self-renewal. According to some embodiments, cancer stem cell or cancer stem cells refer to a minute population of cancer stem cells that have self-renewal capability and are tumorigenic.

Disclosed herein are methods of inhibiting, reducing, and/or diminishing cancer stem cell survival and/or self-renewal comprising administering a therapeutically effective amount of at least one compound of formula (I) in combination with a therapeutically effective amount of at least one gemcitabine. Disclosed herein are methods of inhibiting, reducing, and/or diminishing cancer stem cell survival and/or self-renewal comprising administering a therapeutically effective amount of at least one compound of formula (I) in combination with a therapeutically effective amount of at least one nab-paclitaxel. Also disclosed herein are methods of inhibiting, reducing, and/or diminishing cancer stem cell survival and/or self-renewal comprising administering a therapeutically effective amount of at least one compound of formula (I) in combination with a therapeutically effective amount of at least one gemcitabine and a therapeutically effective amount of at least one nab-paclitaxel. In some embodiments, the at least one compound of formula (I) is included in a pharmaceutical composition.

Disclosed herein are methods of treating at least one cancer that is refractory to conventional chemotherapies and/or targeted therapies in a subject comprising administering a therapeutically effective amount of at least one compound of formula (I) in combination with a therapeutically effective amount of at least one gemcitabine. Also disclosed herein are methods of treating at least one cancer that is refractory to conventional chemotherapies and/or targeted therapies in a subject comprising administering a therapeutically effective amount of at least one compound of formula (I) in combination with a therapeutically effective amount of at least one nab-paclitaxel. Disclosed herein are methods of treating at least one cancer that is refractory to conventional chemotherapies and/or targeted therapies in a subject comprising administering a therapeutically effective amount of at least one compound of formula (I) in combination with a therapeutically effective amount of at least one gemcitabine and a therapeutically effective amount of at least one nab-paclitaxel. In various embodiments, the at least one compound of formula (I) is included in a pharmaceutical composition.

Disclosed herein are methods of treating recurrent cancer in a subject that has failed surgery, oncology therapy (e.g., chemotherapy), or radiation therapy, comprising administering a therapeutically effective amount of at least one compound of formula (I) in combination with a therapeutically effective amount of at least one gemcitabine. Disclosed herein are methods of treating recurrent cancer in a subject that has failed surgery, oncology therapy (e.g., chemotherapy), or radiation therapy, comprising administering a therapeutically effective amount of at least one compound of formula (I) in combination with a therapeutically effective amount of at least one nab-paclitaxel. Disclosed herein are methods of treating recurrent cancer in a subject that has failed surgery, oncology therapy (e.g., chemotherapy), or radiation therapy, comprising administering a therapeutically effective amount of at least one compound of formula (I) in combination with a therapeutically effective amount of at least one gemcitabine and a therapeutically effective amount of at least one nab-paclitaxel. In various embodiments, the at least one compound of formula (I) is included in a pharmaceutical composition.

Disclosed herein are methods of treating or preventing cancer metastasis in a subject, comprising administering a therapeutically effective amount of at least one compound of formula (I) in combination with a therapeutically effective amount of at least one gemcitabine. Also disclosed herein are methods of treating or preventing cancer metastasis in a subject, comprising administering a therapeutically effective amount of at least one compound of formula (I) in combination with a therapeutically effective amount of at least one nab-paclitaxel. Also disclosed herein are methods of treating or preventing cancer metastasis in a subject, comprising administering a therapeutically effective amount of at least one compound of formula (I) in combination with a therapeutically effective amount of at least one gemcitabine and a therapeutically effective amount of at least one nab-paclitaxel. In various embodiments, the at least one compound of formula (I) is included in a pharmaceutical composition.

Disclosed herein are methods of treating cancer in a subject comprising administering a therapeutically effective amount of at least one compound of formula (I) in combination a therapeutically effective amount of at least one gemcitabine. Disclosed herein are methods of treating cancer in a subject comprising administering a therapeutically effective amount of at least one compound of formula (I) in combination a therapeutically effective amount of at least one nab-paclitaxel. Disclosed herein are methods of treating cancer in a subject comprising administering a therapeutically effective amount of at least one compound of formula (I) in combination a therapeutically effective amount of at least one gemcitabine and a therapeutically effective amount of at least one nab-paclitaxel. In various embodiments, the at least one compound of formula (I) is included in a pharmaceutical composition.

In some embodiments, the cancer may be metastatic pancreatic ductal adenocarcinoma. In some embodiments, the cancer may be refractory. In some embodiments, the cancer may be recurrent. In some embodiments, the cancer may be metastatic. In some embodiments, the cancer may be associated with overexpression of activated pSTAT3. In some embodiments, the cancer may be associated with nuclear β-CATENIN localization.

### EXAMPLES

Examples are provided below to further illustrate different features of the present disclosure. The examples also illustrate useful methodology for practicing the invention. These examples do not limit the claimed invention.

The methods disclosed herein comprise administering to a subject in need thereof comprising a therapeutically effective amount of at least one gemcitabine, at least one nab-paclitaxel, and at least one compound of formula (I).

### Example 1

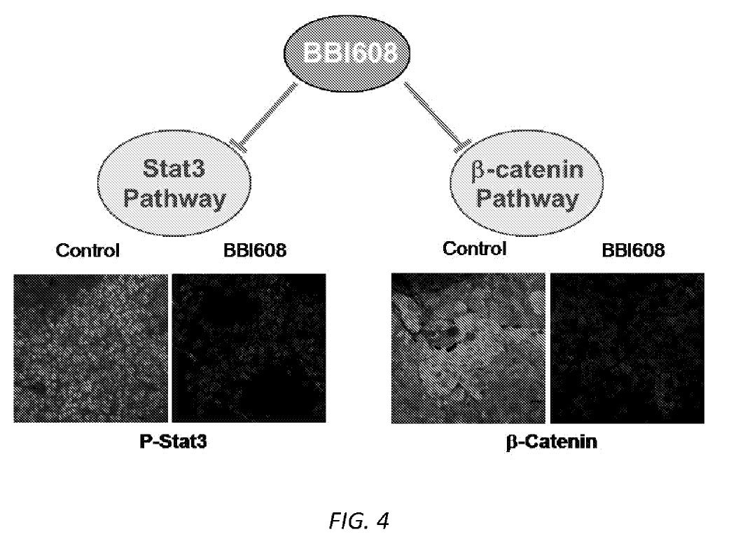

The effects of 2-acetylnaphtho[2,3-b]furan-4,9-dione, a compound of formula (I), to tumor cells were studied by analyzing the tumor cells treated with the compound or without the compound (control) with immunofluorescence staining using antibodies specific for human p-STAT3 and 13-CATENIN. As shown in FIG. 4, tumor cells with positive staining for human p-STAT3 and 13-CATENIN were effectively inhibited by 2-acetylnaphtho[2,3-b]furan-4,9-dione, a compound of formula (I).

### Example 2

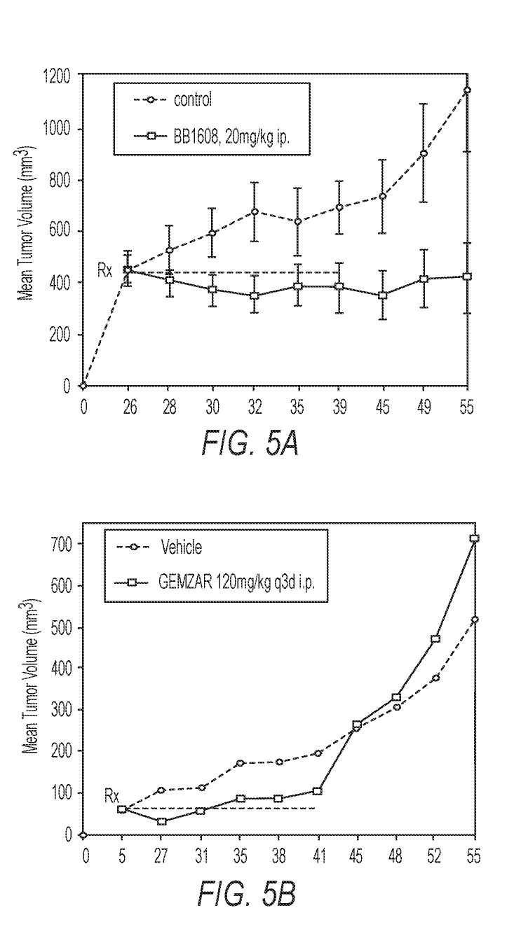

The effects of 2-acetylnaphtho[2,3-b]furan-4,9-dione, a compound of formula (I), on a human PDAC xenograft tumor (Paca-2) in nude mice were studied. Specifically, immunosuppressed mice with Paca-2 human pancreatic cancer were given 2-acetylnaphtho[2,3-b]furan-4,9-dione (20 mg/kg) or vehicle control IP daily or gemcitabine (120 mg/kg) or vehicle control IP every 3 days. Tumor size was evaluated periodically during treatment. Each point represents the mean+SEM of five tumors. As shown in FIGS. 5A and 5B, 2-acetylnaphtho[2,3-b]furan-4,9-dione, a compound of formula (I), was effective at inhibiting tumor growth while gemcitabine showed only a marginal effect on tumor growth.

### Example 3

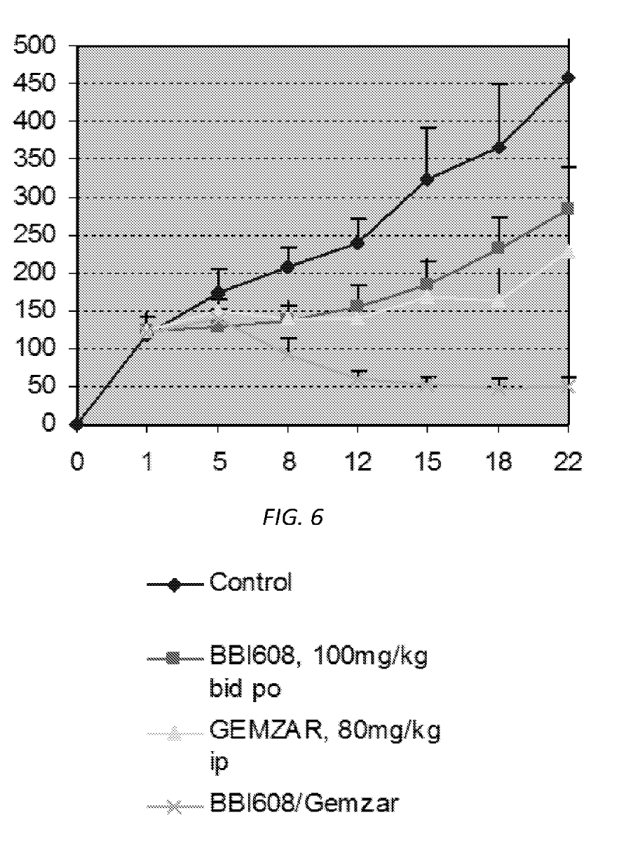

The effects of 2-acetylnaphtho[2,3-b]furan-4,9-dione, a compound of formula (I), gemcitabine, and a combination of 2-acetylnaphtho[2,3-b]furan-4,9-dione and gemcitabine were studied. Specifically, immunosuppressed mice with established human pancreatic adenocarcinoma (Panc-1) were treated with vehicle control, 2-acetylnaphtho[2,3-b]furan-4,9-dione (100 mg/kg, PO, bid), gemcitabine (Gemzar, 80 mg/kg, IV, q3d), or the combination of 2-acetylnaphtho[2,3-b]furan-4,9-dione and gemcitabine. Tumor size was evaluated periodically during treatment. Each point represents the mean+SEM of five tumors. As shown in FIG. 6, while 2-acetylnaphtho[2,3-b]furan-4,9-dione or gemcitabine showed certain effects in inhibiting tumor growth, the combination significantly reduced tumor growth in the mouse model.

### Example 4

The effects of 2-acetylnaphtho[2,3-b]furan-4,9-dione, a compound of formula (I), in combination with gemcitabine and nab-paclitaxel in patients with metastatic pancreatic ductal adenocarcinoma (mPDAC) were studied in a Phase Ib extension, open label, multi-center study.

In that clinical study, the safety, tolerability and the recommended Phase II dose (RP2D), PK profile, and signs of anti-cancer activity of 2-acetylnaphtho[2,3-b]furan-4,9-dione in combination with nab-paclitaxel and gemcitabine were assessed in adult patients with metastatic pancreatic cancer. In each treatment cycle, 2-acetylnaphtho[2,3-b]furan-4,9-dione was administered at 240 mg BID for 4 weeks in combination with nab-paclitaxel 125 mg/m² and gemcitabine 1000 mg/m² administered weekly for 3 out of the 4 weeks. The treatment continued in 28-day cycles.

In addition, the pharmacokinetic profile and the pharmacodynamics (biomarkers) of 2-acetylnaphtho[2,3-b]furan-4,9-dione in combination with gemcitabine and nab-paclitaxel were studied.

In total, 37 patients aged 46-79 with histologically or cytologically confirmed metastatic pancreatic adenocarcinoma for which gemcitabine and nab-paclitaxel are acceptable therapeutic options were enrolled in the open label, multi-center, phase Ib study (see Table 2). Of the 37 patients, 29 patients (78%) were treatment-naïve and 8 patients (22%) had received neoadjuvant systemic therapy and 5 patients (14%) were previously exposed to gemcitabine treatment.

TABLE 2
Baseline Demographics & Laboratory Values (N = 37).

| Prior Adjuvant Rx | N | % |
|---|---|---|
| Treatment naïve | 29 | 78 |
| Any adjuvant Rx | 8 | 22 |
| Prior Gemcitabine | 5 | 14 |
| Age | | |
| Median | 63 | yrs |
| Range | 46-79 | yrs |
| Gender | | |
| Female | 20 | 54% |
| Male | 17 | 46% |
| Race | | |
| Caucasian | 34 | 92% |
| Black | 2 | 5% |
| Asian | 1 | 3% |
| — | | |
| ECOG | | |
| 0 | 16 | 43% |
| 1 | 21 | 57% |
| Hemoglobin | | |
| Median | 129 | g/L |
| Range | 99-154 | g/L |
| Neutrophils | | |
| Median | 5 | 10⁹/L |
| Range | 0.8-10.5 | 10⁹/L |
| Platelets | | |
| Median | 224 | 10⁹/L |
| Range | 109-564 | 10⁹/L |
| ALT | | |
| Median | 32 | U/L |
| Range | 12-152 | U/L |
| AST | | |
| Median | 27 | U/L |
| Range | 11-101 | U/L |
| Creatinine | | |
| Median | 72.5 | μMOL/L |
| Range | 30.9-114 | μMOL/L |

The patients received continuous oral administration of BBI-608 twice daily in 28 day cycles. A standard gemcitabine and nab-paclitaxel regimen was administered on days 1, 8 and 15 of every 28 day study cycle. Specifically, BBI-608 was administered at 240 mg BID in combination with gemcitabine 1000 mg/m² and nab-paclitaxel 125 mg/m² administered weekly for 3 out every 4 weeks until progression of disease, unacceptable toxicity, or another discontinuation criterion was met. Pharmacokinetics and pharmacodynamics were evaluated and objective tumor response was assessed every 8 weeks using Response Evaluation Criteria In Solid Tumors (RECIST 1.1).

Of the 37 patients enrolled, 30 were evaluable for response.

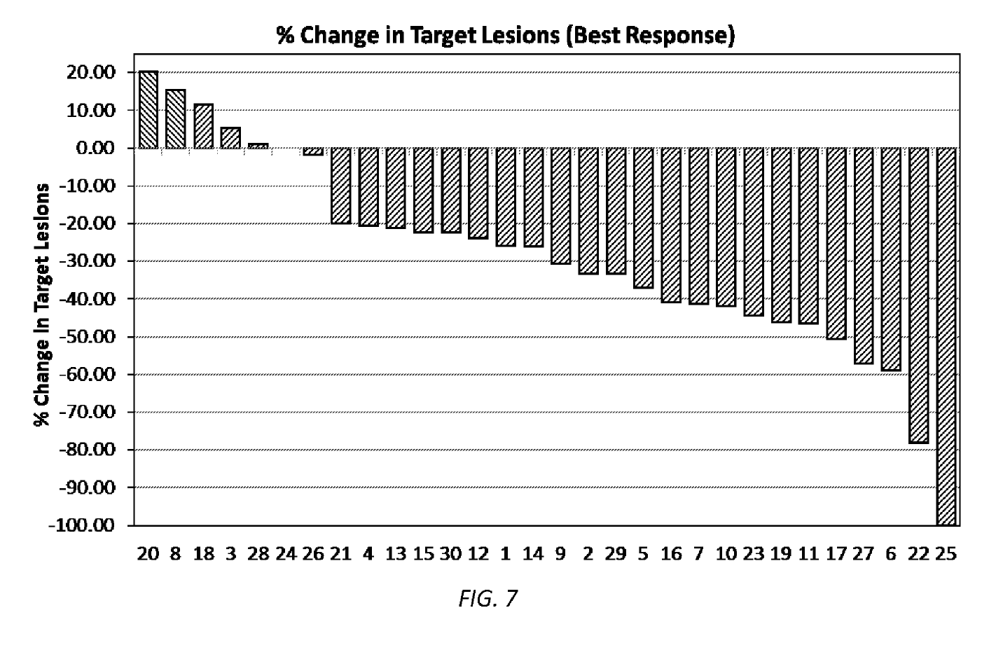

Anti-cancer activity was observed in patients with mPDAC (see, FIG. 7). For example, as shown in Table 3 and FIG. 7, disease control (CR+PR+SD) was observed in 28 of 30 evaluable patients (93%), with 24 patients (80%) with tumor regression of which 1 patient achieved CR (3%) and 14 patients (47%) achieved PR (RECIST 1.1: 31-100.0% regression). Of the 7 patients who were non-evaluable for treatment response, 3 stopped treatment due to clinical progression, 1 stopped treatment due to non-compliance, and 3 withdrew consent. Among 37 patients enrolled (intent-to-treat), disease control (CR+PR+SD) was observed in 28 pts (76%), with tumor regression observed in 24 patients (65%) of which 1 patient achieved CR (3%) and 14 patients achieved PR (38%).

This study demonstrated that 2-acetylnaphtho[2,3-b]furan-4,9-dione (240 mg BID q12 hours) combined with gemcitabine with nab-paclitaxel at full doses effectively promoted anti-tumor activity.

TABLE 3
(Patient Summary).

| | | Best | |
|---|---|---|---|
| | Weeks | Response | |
| Patient | on | (RECIST | |
| # | Study | 1.1) | Comment |
| 1 | 34.0 | SD | 25.8% tumor regression, prolonged SD |
| 2 | 32.9 | PR | 33.3% tumor regression, prolonged SD/PR |
| 3 | 4.7 | SD | 5.5% tumor growth, prolonged SD* |
| 4 | 50.0 | SD | 20.5% tumor regression, prolonged SD |
| 5 | 39.7 | PR | 37.1% tumor regression, prolonged SD/PR |
| 6 | 62+ | PR | 58.9% tumor regression, prolonged |
| | | | SD/PR; continuing on study |
| 7 | 19.4 | PR | 41.3% tumor regression |
| 8 | 8.9 | PD | 15.3% tumor growth |
| 9 | 40.0 | PR | 30.6% tumor regression; prolonged SD/PR |
| 10 | 30.7 | PR | 41.9% tumor regression; prolonged SD/PR |
| 11 | 24.7 | PR | 46.4% tumor regression |
| 12 | 47+ | SD | 23.8% tumor regression; prolonged SD; |
| | | | continuing on study |
| 13 | 48+ | SD | 21.1% tumor regression; prolonged SD; |
| | | | continuing on study |
| 14 | 16.9 | SD | 26.1% tumor regression |
| 15 | 22.3 | SD | 22.2% tumor regression |
| 16 | 9.6 | PR | 40.8% tumor regression |
| 17 | 39.7 | PR | 50.6% tumor regression; prolonged PR |
| 18 | 18.1 | SD | 11.6% tumor growth |
| 19 | 24.9 | PR | 46.2% tumor regression |
| 20 | 9.0 | PD | 20.3% tumor growth |
| 21 | 12.9 | SD | 19.9% tumor regression |
| 22 | 31.3 | PR | 78.1% tumor regression; prolonged PR |
| 23 | 37+ | PR | 44.3% tumor regression; prolonged |
| | | | SD/PR; continuing on study |
| 24 | 6.9 | SD | No change |
| 25 | 34+ | CR | 100.0% tumor regression; prolonged |
| | | | SD/CR; continuing on study |
| 26 | 16.9 | SD | 1.7% tumor regression |
| 27 | 32+ | PR | 57.1% tumor regression; prolonged |
| | | | SD/PR; continuing on study |
| 28 | 4.9 | SD | 1.1% tumor growth |
| 29 | 31+ | PR | 33.3% tumor regression; prolonged |
| | | | SD/PR; continuing on study |
| 30 | 28+ | SD | 22.2% tumor regression; prolonged SD; |
| | | | continuing on study |

* Patient maintained SD after discontinuation from protocol treatment
CR: complete response;
PR: partial response;
SD: stable disease;
RECIST: Response Evaluation Criteria In Solid Tumors

The combination of 2-acetylnaphtho[2,3-b]furan-4,9-dione, gemcitabine, and nab-paclitaxel was well tolerated with no new adverse events observed beyond what is typically associated with gemcitabine and nab-paclitaxel. This combination treatment had no observed dose-limiting toxicity and a safety profile similar to that of each agent individually. No significant pharmacokinetic interactions were observed.

The majority of adverse events observed were grade 1 or 2 gastrointestinal adverse events (see Table 4). For example, most common adverse events (AEs) related to napabucasin included grade 1 diarrhea, abdominal pain, nausea, and fatigue with grade 3 AEs observed in 9 patients: 5 patients (fatigue), 1 patient (diarrhea), 1 patient (dehydration), 1 patient (nausea) and 1 patient (hypokalemia) (see Table 4). No significant pharmacokinetic interactions were observed. The gastrointestinal adverse events may be easily manageable with anti-diarrheals and anti-emetic supportive medications.

TABLE 4
(Adverse Events (possibly/probably/definitely related to Napabucasin and/or
nab-PTX and/or gemcitabine), any grade ≥10%, N = 37 as of May 31,
2016). Number and Percent of Total Subjects with a given Adverse Event by Grade

| | | Grade 1 | | Grade 2 | | Grade 3 | | Grade 4 | |
|---|---|---|---|---|---|---|---|---|---|
| Organ System | Event | # | % | # | % | # | % | # | % |
| Gastrointestinal | Diarrhea | 26 | 70.3% | 7 | 18.9% | 1 | 2.7% | 0 | 0.0% |
| | Nausea | 15 | 40.5% | 3 | 8.1% | 1 | 2.7% | 0 | 0.0% |
| | Abdominal Pain | 10 | 27.0% | 2 | 5.4% | 0 | 0.0% | 0 | 0.0% |
| | Vomiting | 9 | 24.3% | 2 | 5.4% | 0 | 0.0% | 0 | 0.0% |
| Constitutional | Fatigue | 17 | 45.9% | 10 | 27.0% | 7 | 18.9% | 0 | 0.0% |
| | Fever | 9 | 24.3% | 0 | 0.0% | 0 | 0.0% | 1 | 2.7% |
| | Edema Limbs | 8 | 21.6% | 3 | 8.1% | 0 | 0.0% | 0 | 0.0% |
| | Platelet Count Decreased | 7 | 18.9% | 3 | 8.1% | 4 | 10.8% | 0 | 0.0% |
| | White Blood Cell Decreased | 4 | 10.8% | 5 | 13.5% | 3 | 8.1% | 1 | 2.7% |
| | Lymphocyte Count Decreased | 3 | 8.1% | 3 | 8.1% | 3 | 8.1% | 1 | 2.7% |
| Skin And | Alopecia | 6 | 16.2% | 6 | 16.2% | 0 | 0.0% | 0 | 0.0% |
| Subcutaneous | Rash Maculo-Papular | 5 | 13.5% | 0 | 0.0% | 0 | 0.0% | 0 | 0.0% |
| Tissue | | | | | | | | | |
| Hematologic | Anemia | 7 | 18.9% | 9 | 24.3% | 4 | 10.8% | 0 | 0.0% |
| | Neutropenia | 3 | 8.1% | 3 | 8.1% | 6 | 16.2% | 1 | 2.7% |
| Metabolism | Anorexia | 8 | 21.6% | 2 | 5.4% | 0 | 0.0% | 0 | 0.0% |
| And Nutrition | | | | | | | | | |
| Neuro- | Dysgeusia | 5 | 13.5% | 0 | 0.0% | 0 | 0.0% | 0 | 0.0% |
| Psychiatric | | | | | | | | | |
| Pulmonary | Dyspnea | 4 | 10.8% | 1 | 2.7% | 0 | 0.0% | 0 | 0.0% |
| Infections And | Mucosal Infection | 3 | 8.1% | 1 | 2.7% | 0 | 0.0% | 0 | 0.0% |
| Infestations | | | | | | | | | |
| Renal And | Urine Discoloration | 4 | 10.8% | 0 | 0.0% | 0 | 0.0% | 0 | 0.0% |
| Urinary | | | | | | | | | |
| Other | Neuropathy | 13 | 35.1% | 5 | 13.5% | 4 | 10.8% | 0 | 0.0% |
| | Rash | 5 | 13.5% | 1 | 2.7% | 0 | 0.0% | 0 | 0.0% |

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described accordingly, all suitable modifications and equivalents may be resorted to, falling within the scope of the present disclosure.

1. A method for treating cancer in a subject comprising administering to a subject in need thereof a therapeutically effective amount of at east one compound of formula (I): 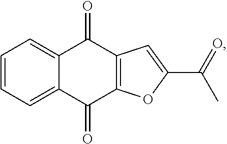 [SMILES: CC(=O)c1cc2c(o1)C(=O)c1ccccc1C2=O]
or a prodrug, pharmaceutically acceptable salt of any of the foregoing, or solvate of any of the foregoing,
a therapeutically effective amount of at least one gemcitabine, and/or
a therapeutically effective amount of at least one nab-paclitaxel.

2. The method according to claim 1, wherein the cancer progressed on at least one prior chemotherapy regimen.

3. A method for resensitizing a subject to a prior chemotherapy regimen comprising administering to a subject whose cancer progressed on a prior chemotherapy a therapeutically effective amount of at least one compound of formula (I): 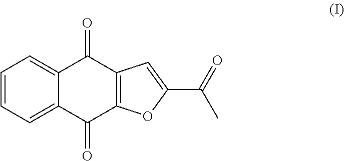 [SMILES: CC(=O)c1cc2c(o1)C(=O)c1ccccc1C2=O]
or a prodrug, pharmaceutically acceptable salt of any of the foregoing, or solvate of any of the foregoing.

4. A method of simultaneously inhibiting, reducing, and/or diminishing survival and/or self-renewal of cancer stem cells, and inhibiting, reducing, and/or diminishing survival and/or proliferation of heterogeneous cancer cells chosen from pancreatic cancer cells in a subject having cancer, comprising administering to a subject in need thereof:
a therapeutically effective amount of at least one compound of formula (I) 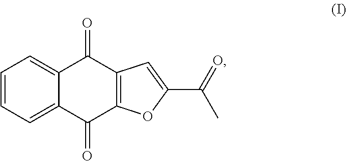 [SMILES: CC(=O)c1cc2c(o1)C(=O)c1ccccc1C2=O]
or a prodrug, pharmaceutically acceptable salt of any of the foregoing, or solvate of any of the foregoing,
a therapeutically effective amount of at least one gemcitabine, and/or
a therapeutically effective amount of at least one nab-paclitaxel.

5. A method preventing cancer relapses in a subject comprising administering to the subject:
a therapeutically effective amount of at least one compound of formula (I): 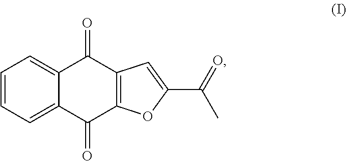 [SMILES: CC(=O)c1cc2c(o1)C(=O)c1ccccc1C2=O]
or a prodrug, pharmaceutically acceptable salt of an of the foregoing, or solvate of any of the foregoing,
a therapeutically effective amount of at least one gemcitabine, and/or
a therapeutically effective amount of at least one nab-paclitaxel.

6. (canceled)

7. The method according to any one of claims 1 and 3-5, wherein the cancer is pancreatic cancer.

8. The method according to claim 7, wherein the pancreatic cancer is metastatic pancreatic ductal adenocarcinoma.

9. The method according to any one of claims 1 and 3-5, wherein the at least one compound of formula (I) is administered at a dose of about 480 mg per day.

10. The method according to claim 9, wherein the at least one compound of formula (I) is administered in a divided dose.

11. The method according to any one of claims 1 and 3-5, wherein the at least one compound of formula (I) is administered at a dose of about 240 mg twice daily.

12. The method according to any one of claims 1 and 3-5, wherein the patient is pre-treated with a standard chemotherapy.

13. The method according to any one of claims 1 and 3-5, wherein the at least one gemcitabine is administered as a weekly infusion of about 1000 mg gemcitabine/m².

14. The method according to any one of claims 1 and 3-5, wherein the at least one nab-paclitaxel is administered as weekly infusion of about 125 mg gemcitabine/m².

15. The method according to any one of claims 1 and 3-5, wherein the cancer is advanced, metastatic, unresectable, or recurrent.

16. A kit comprising at least one compound of formula (I): 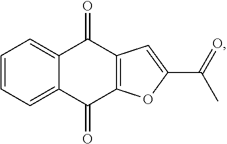 [SMILES: CC(=O)c1cc2c(o1)C(=O)c1ccccc1C2=O]
at least one gemcitabine and/or;
at least one nab-paclitaxel.
